(19) United States
(12) Patent Application Publication
(10) Pub. No.: US 2018/0169366 A1
(43) Pub. Date: Jun. 21, 2018

(54) CUSHION COVER AND PATIENT INTERFACE DEVICE USING SAME

(71) Applicant: KONINKLIJKE PHILIPS N.V., EINDHOVEN (NL)

(72) Inventors: ADAM LEVERN BELL, PITTSBURGH, PA (US); DANIEL STEED, NORTH HUNTINGDON, PA (US)

(21) Appl. No.: 15/578,732

(22) PCT Filed: May 19, 2016
(86) PCT No.: PCT/IB2016/052928

§ 371 Date: Dec. 1, 2017

Related U.S. Application Data
(60) Provisional application No. 62/171,262, filed on Jun. 5, 2015.

(51) Int. Cl. A61M 16/06

(52) U.S. Cl. CPC A61M 16/0622; A61M 16/0633; A61M 2207/00; A61M 2205/588; A61M 2205/0238

(57) ABSTRACT

A cushion cover (20) for a patient interface device (8) having a cushion (12) structured to receive a flow of treatment gas. The cushion cover includes a first portion (22) structured to conform to shapes of back surfaces (32) of the cushion and a portion of the side surfaces (34) of the cushion, a second portion (24) structured to conform to shapes of a remainder of the side surfaces of the cushion, and an opening (25) structured to allow treatment gas to pass therethrough, wherein the cushion cover is structured to fit over the cushion.

### CROSS-REFERENCE TO RELATED APPLICATIONS

This patent application claims the priority benefit under 35 U.S.C. §119(e) of U.S. Provisional Application No. 62/171,262, filed on Jun. 5, 2015, the contents of which are herein incorporated by reference.

### BACKGROUND OF THE INVENTION

### 1. Field of the Invention

The present invention pertains to patient interface devices structured to deliver a flow of breathing gas to a patient, and, in particular, to cushion covers for use with patient interface devices.

### 2. Description of the Related Art

Obstructive sleep apnea (OSA) is a condition that affects millions of people from around the world. OSA is characterized by disturbances or cessation in breathing during sleep. OSA episodes result from partial or complete blockage of airflow during sleep that lasts at least 10 seconds and often as long as 1 to 2 minutes. In a given night, people with moderate to severe apnea may experience complete or partial breathing disruptions as high as 200-500 per night. Because their sleep is constantly disrupted, they are deprived of the restorative sleep necessary for efficient functioning of body and mind. This sleep disorder has also been linked with hypertension, depression, stroke, cardiac arrhythmias, myocardial infarction and other cardiovascular disorders. OSA also causes excessive tiredness. p Non-invasive ventilation and pressure support therapies involve the placement of a patient interface device, which is typically a nasal mask, a nasal/oral mask (also referred to as a full face mask), or a pillows type mask, on the face of a patient to interface the ventilator or pressure support system with the airway of the patient so that a flow of breathing gas can be delivered from the pressure/flow generating device to the airway of the patient.

Typically, patient interface devices include a mask shell or frame having a cushion attached to the shell that contacts the surface of the patient. The mask shell and cushion are held in place by a headgear that wraps around the head of the patient. The mask and headgear form the patient interface assembly. A typical headgear includes flexible, adjustable straps that extend from the mask to attach the mask to the patient.

Because patient interface devices are typically worn for an extended period of time, a variety of concerns must be taken into consideration. For example, in providing CPAP to treat OSA, the patient normally wears the patient interface device all night long while he or she sleeps. One concern in such a situation is that the patient interface device is as comfortable as possible, otherwise the patient may avoid wearing the interface device, defeating the purpose of the prescribed pressure support therapy. Wearing a patient interface device for a prolonged period of time can also irritate and cause red marks to form on a patient's skin. The irritation and red marks may arise due to a variety of causes such as friction, tension or sweat. There is also a concern that silicone materials, which are commonly used in patient interface devices, may exacerbate these causes.

One type of patient interface device uses fabric to cover hard plastic straps on the patient interface device. However, there are still many areas of the patient interface device that are made of plastic or silicone that contact the patient's skin. Another type of patient interface device uses a fabric mask and cushion. In this type of patient interface device, the structure of the mask and cushion are weak compared to those that use silicone or plastic for the mask and cushion. The weak structural support can lead to excess deformation, which in turn may lead leaks or discomfort.

In some other types of patient interface devices, a separate fabric accessory is used in the area between the cushion and the patient's skin. One type of such a fabric accessory is a piece of fabric that is placed between the cushion and the patient's face when the patient interface device is worn. However, the fabric accessory does not actually attach to the patient interface device. The fabric accessory is thus prone to being misplaced or moving while the patient interface device is in use. Another type of fabric accessory is a sleeve that fits around the mask and cushion of the patient interface device. While sleeve is attached to the mask and cushion, it is still prone to slipping and wrinkling.

### SUMMARY OF THE INVENTION

In one embodiment, a cushion cover for a patient interface device having a cushion structured to receive a flow of treatment gas comprises a first portion structured to conform to shapes of back surfaces of the cushion and a portion of the side surfaces of the cushion, a second portion structured to conform to shapes of a remainder of the side surfaces of the cushion, and an opening structured to allow treatment gas to pass therethrough, wherein the cushion cover is structured to fit over the cushion.

In another embodiment, a patient interface device comprises a cushion structured to receive a flow of treatment gas and a cushion cover including a first portion structured to conform to shapes of back surfaces of the cushion and a portion of the side surfaces of the cushion, a second portion structured to conform to shapes of a remainder of the side surfaces of the cushion, and an opening structured to allow treatment gas to pass therethrough, wherein the cushion cover is structured to fit over the cushion.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

### BRIEF DESCRIPTION OF THE DRAWINGS

FIG. 1 is a schematic view of a system adapted to provide a regiment of respiratory therapy to a patient according to one exemplary embodiment of the disclosed concept;

FIG. 2 is a schematic view of the system of FIG. 1 with a cushion cover separated from a patient interface device;

FIG. 3 is a front view of a cushion cover in accordance with an exemplary embodiment of the disclosed concept;

FIG. 4 is a side view of the cushion cover of FIG. 3;

FIG. 5 is a rear view of the cushion cover of FIG. 3;

### DETAILED DESCRIPTION OF EXEMPLARY EMBODIMENTS

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and then coupled together as a unit is not a “unitary” component or body. As employed herein, the statement that two or more parts or components “engage” one another shall mean that the parts exert a force against one another either directly or through one or more intermediate parts or components. As employed herein, the term “number” shall mean one or an integer greater than one (i.e., a plurality).

The terms “front side,” “front surface” or the like, when used with reference to a patient interface device or components of a patient interface device refer to sides or surfaces that face in a direction substantially away from a patient's face when the patient interface device is worn by the patient.

The terms “back side,” “back surface,” “rear side,” “rear surface” or the like, when used with reference to a patient interface device or components of a patient interface device refer to sides or surfaces that face in a direction substantially toward a patient's face when the patient interface device is worn by the patient.

The terms “side surface” or the like, when used with reference to a patient interface device or components of a patient interface device refer to sides or surfaces that connect the front and back surfaces.

As used herein, the term “fabric” shall mean a material consisting of a network of interlaced or otherwise entangled natural or artificial fibers made by, for example and without limitation, weaving, knitting, spreading, crocheting, or bonding (e.g., by chemical, mechanical, heat or solvent treatment) the fibers to form the network, and may include, for example, and without limitation, woven and nonwoven fabric materials.

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

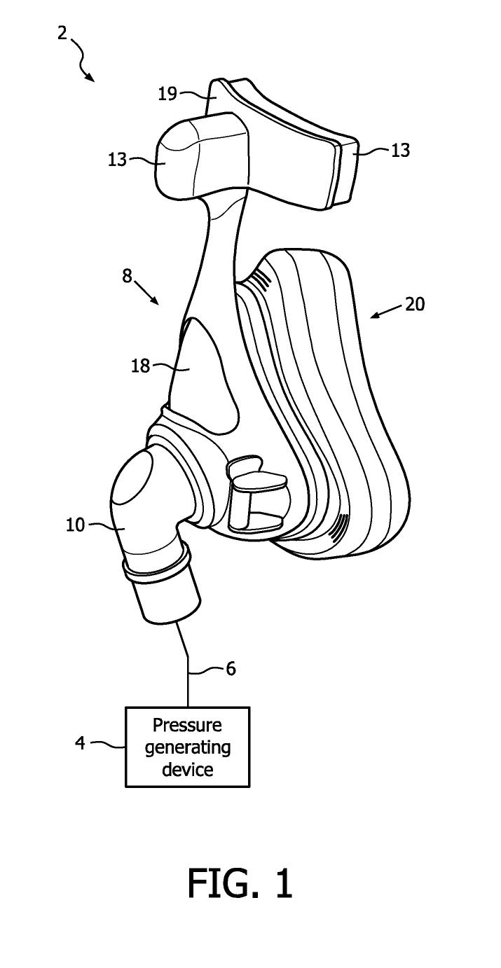

A system 2 adapted to provide a regimen of respiratory therapy to a patient according to one exemplary embodiment of the disclosed concept is generally shown in FIG. 1. System 2 includes a pressure generating device 4, a delivery conduit 6, and a patient interface device 8 including an elbow connector 10 fluidly coupled to delivery conduit 6.

Pressure generating device 4 is structured to generate a flow of breathing gas and may include, without limitation, ventilators, constant pressure support devices (such as a continuous positive airway pressure device, or CPAP device), variable pressure devices (e.g., BiPAP®, Bi-Flex®, or C-Flex™ devices manufactured and distributed by Philips Respironics of Murrysville, Pa.), and auto-titration pressure support devices. Delivery conduit 6 is structured to communicate the flow of breathing gas from pressure generating device 4 to patient interface device 8.

In the illustrated embodiment, patient interface 8 is a nasal/oral mask structured to cover the nose and mouth of the patient. However, any other type of patient interface device 8 which facilitates the delivery of the flow of breathing gas to, and the removal of a flow of exhalation gas from, the airway of a patient may be used, such as, without limitation, a nasal mask that covers the patient's nose or a pillows type mask, while remaining within the scope of the present disclosed concept. In the embodiment shown in FIG. 1, patient interface device 8 includes a rigid or semi-rigid mask 18 and a flexible cushion (hidden behind cushion cover 20). An opening in mask 18 to which elbow connector 10 is coupled allows the flow of breathing gas from pressure generating device 4 to be communicated to an interior space defined by mask 18 and cushion, and then, to the airway of a patient. Mask 18 also includes a forehead portion 19 that is structured to contact the patient's forehead area via a forehead cushion 13.

Straps (not shown) may be attached to mask 18 to secure patient interface device 8 to the patient's head. Straps may be adjusted to tighten or loosen the fit of patient interface device 8 to the patient.

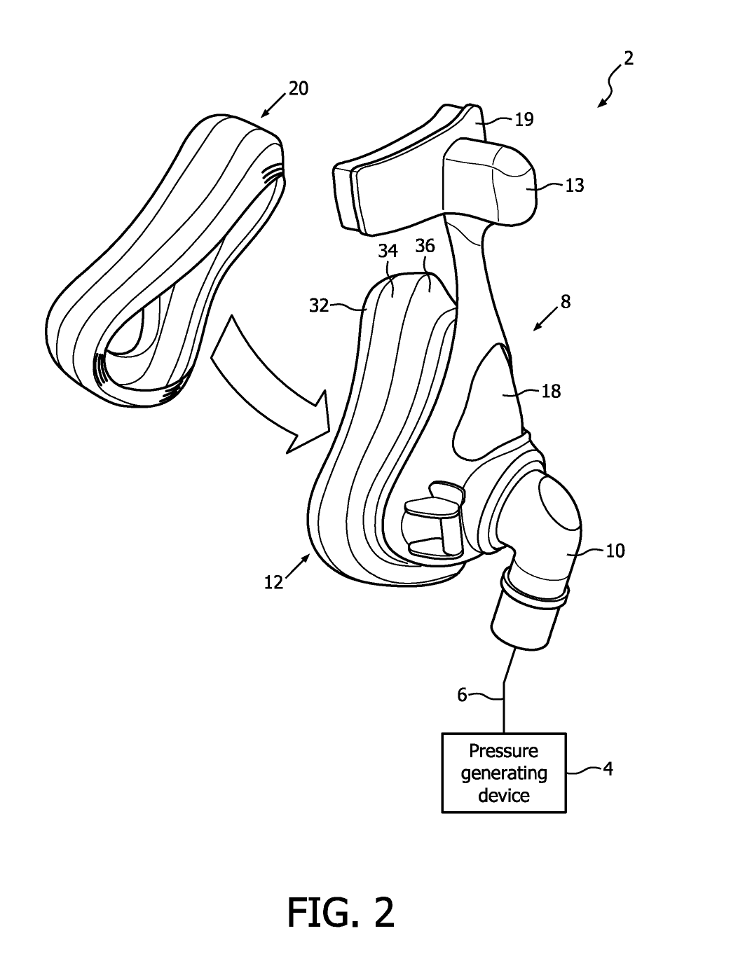

Cushion cover 20 is a removable piece that is structured to fit over the cushion 12 (see FIG. 2) of patient interface device 8. Cushion cover 20 in accordance with embodiments of the disclosed concept will be described in more detail hereinafter.

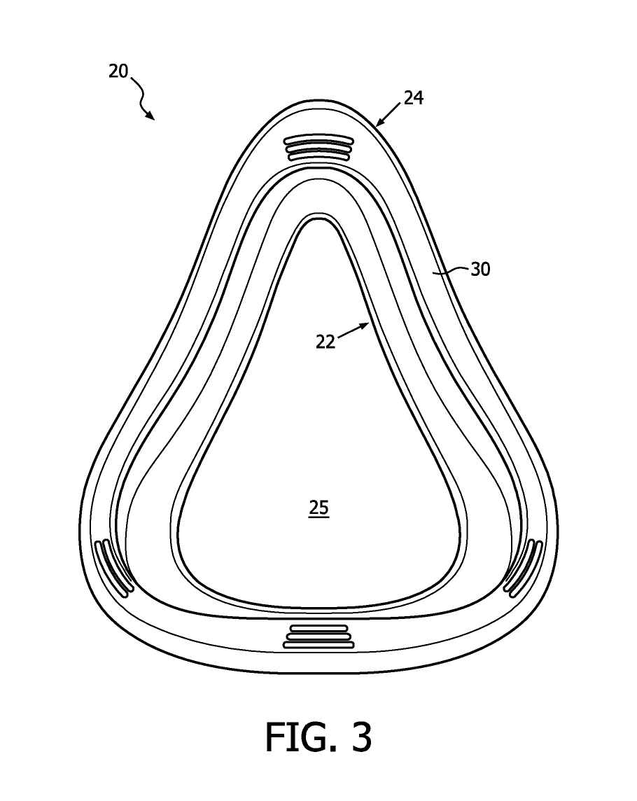

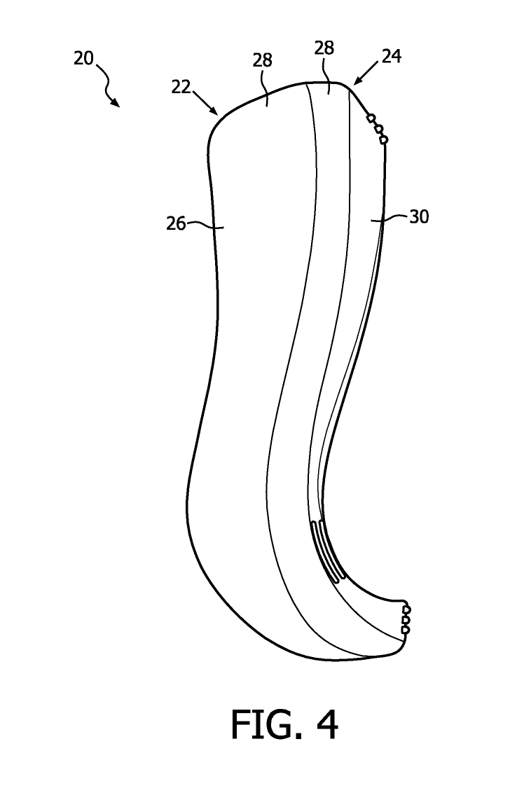

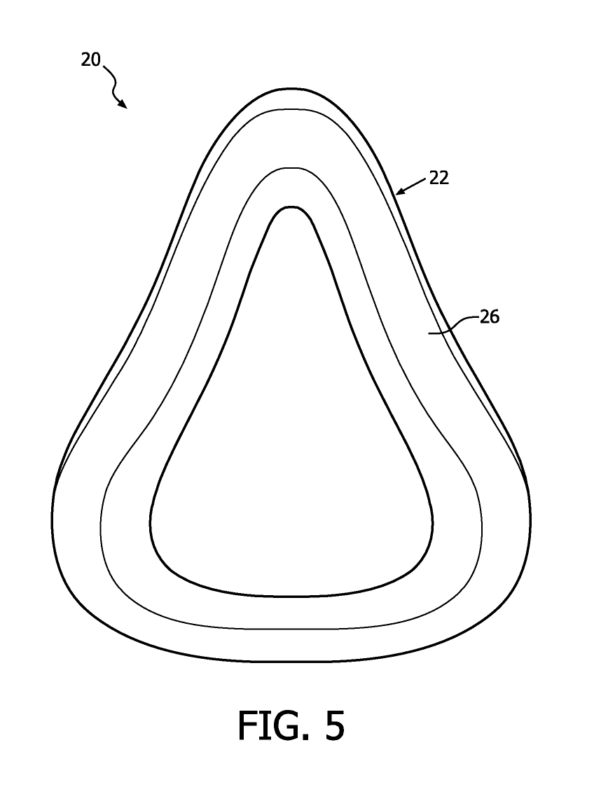

FIGS. 3, 4 and 5 are front, side and rear views, respectively, of cushion cover 20 in accordance with an example embodiment of the disclosed concept. Cushion cover 20 may be employed in conjunction with patient interface device 8 of FIGS. 1 and 2. Cushion cover 20 includes a first portion 22 and a second portion 24. First portion 22 includes the back surfaces 26 and a portion of the side surfaces 28 of cushion cover 20. Back surfaces 26 of cushion cover 20 are structured to contact a patient's face when cushion cover 20 is fitted over cushion 22 of patient interface device 8. Second portion 24 of cushion cover 20 includes the remainder of the side surfaces 28 and the front surfaces 30 of cushion cover 20.

Cushion cover 20 has a shape that substantially conforms to the shape of cushion 12. That is, a shape of first portion 22 conforms to the corresponding shape of back surfaces 32 and a portion of the side surfaces 34 of cushion 12 and a shape of second portion 24 corresponds to the corresponding shape of the remainder of side surfaces 34 and the front surfaces 36 of cushion 12 (see FIG. 2). Cushion cover 20 is structured to fit over cushion 12 and the shape of cushion cover 20 conforming to the shape of cushion 12 allows cushion cover 20 to securely fit over cushion 12.

Cushion cover 20 may employ material having any suitable durometer or materials having different durometers. In some exemplary embodiments of the disclosed concept, cushion cover 20 employs material, such as, without limitation, silicone, having a durometer in a range of about 20 shore A to about 60 shore A. In some exemplary embodiments of the disclosed concept, different parts of cushion cover 20 have different durometers which allows support areas to be harder and flap or sealing areas to be softer.

Cushion cover 20 also includes an opening 25 formed in a central area of cushion cover 20. Opening 25 corresponds with a similar opening in cushion 12 which allows treatment gas to pass through the cushion 12 to the patient. Opening 25 in cushion cover 20 similarly allows treatment gas to pass through it to the patient.

The front surfaces 30 of second portion 24 of cushion cover 20 form a lip which fits over front surfaces 36 of cushion 12. The lip assists in securing cushion cover 20 in place over cushion 22 so that it does not slip or come loose when patient interface device 8 is worn by a patient.

First portion 22 and second portion 24 of cushion cover 20 may be composed of the same or different materials. In some exemplary embodiments of the disclosed concept, first portion 22 and second portion 24 are composed of the same material and form a unitary member. In some other exemplary embodiments of the disclosed concept, first portion 22 and second portion 24 are composed of different materials.

In one exemplary embodiment of the disclosed concept, first portion is composed of an air-tight material such as, without limitation, a silicone material whose exterior surface is coated with a fabric material. A polyester blend type fabric may be employed as the fabric material. However, it is contemplated that any suitable type of fabric material may be employed without departing from the scope of the disclosed concept.

In this and other exemplary embodiments of the disclosed concept, the second portion may be composed of a silicone material. The silicone material provides structural support to the cushion cover 20. The structural support helps to prevent the cushion cover 20 from becoming wrinkled or otherwise slipping or loosening from the cushion 12.

In another example embodiment of the disclosed concept, first portion 22 is composed of a silicone material including a hydrophilic additive. By including hydrophilic additive material to the silicone material, the first portion 22 is better able to wick moisture away from the patient's skin and thus the likelihood of the first portion 22 causing red marks on the patient's skin is reduced.

In this embodiment, the second portion 24 may be composed of the silicone material including the hydrophilic additive, similar to the first portion 22, or the second portion may be composed of the silicone material without the hydrophilic additive.

In yet another example embodiment of the disclosed concept, first portion 22 is composed of a silicone material with a wrinkle-reducing additive material. The wrinkle-reducing additive material can have an effect of reducing a patient's wrinkles which would encourage the patient to use the cushion cover 20 and associated patient interface device 8. In some example embodiments of the disclosed concept, a seaweed based extract which reduces wrinkles is employed in first potion 22 of cushion cover 20.

In this embodiment, the second portion may be composed of the silicone material including the wrinkle-reducing additive material, similar to the first portion, or the second portion may be composed of the silicone material without the wrinkle-reducing additive material.

In a further exemplary embodiment of the disclosed concept, the first portion is composed of a silicone material with a scented additive material. The scented additive material can produce a pleasurable odor, scent, aroma or the like making it more likely that the patient will use cushion cover 20. This additive material can be provided on the cushion, imbedded in the cushion, or a combination thereof.

In this embodiment, the second portion may be composed of the silicone material including the scented additive material, similar to the first portion, or the second portion may be composed of the silicone material without the scented additive material.

Cushion cover 20 may be manufactured using any suitable manufacturing process. For example and without limitation, first portion 22 may be molded first using any suitable molding process. Second portion 24 may then be molded to first portion 22 using any suitable multi-part molding process such as, without limitation, over-molding or two-shot injection molding. In some exemplary embodiments where first portion 22 and second portion 24 form a unitary member, cushion cover 20 may be formed using a single shot molding process. In some exemplary embodiments where first portion 22 includes a fabric coating, the fabric may be coated onto first portion 22 and then excess fabric may be trimmed to create opening 25. While these are a few examples of processes that may be employed to manufacture cushion cover 20, it will be appreciated by those having ordinary skill in the art that any suitable manufacturing processes may be employed to manufacture cushion cover 20.

Referring to FIG. 1, although a nasal/oral type patient interface device 8 is disclosed, it is contemplated that disclosed concept may be applied to other types of patient interface devices such as, without limitation, nasal type patient interface devices. For example and without limitation, a cushion cover 20 may be shaped to correspond with a cushion of a nasal type patient interface device, or other types of patient interface devices, without departing from the scope of the disclosed concept.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

1. A cushion cover for a patient interface device having a cushion structured to receive a flow of treatment gas, the cushion cover comprising:
a first portion structured to conform to shapes of back surfaces of the cushion and a portion of the side surfaces of the cushion;
a second portion structured to conform to shapes of a remainder of the side surfaces of the cushion and including a lip structured to conform to shapes of front surfaces of the cushion; and
an opening structured to allow treatment gas to pass therethrough, wherein the cushion cover is structured to fit over the cushion, wherein the first portion and the second portion each include material having a durometer of at least 20 shore A, and wherein the first and second portions are structured to retain shapes conforming to surfaces of the cushion when not attached to the cushion.

2. The cushion cover of claim 1, wherein the first portion is includes an air-tight material having an exterior surface coated with fabric.

3. The cushion cover of claim 2, wherein the air-tight material includes a silicone material.

4. The cushion cover of claim 1, wherein the first portion and the second portion form a unitary piece.

5. The cushion cover of claim 1, wherein the first portion includes a hydrophilic material.

6. The cushion cover of claim 1, wherein the first portion includes a wrinkle-reducing material.

7. The cushion cover of claim 1, wherein the first portion includes a scented material.

8. (canceled)

9. A patient interface device comprising:
a cushion structured to receive a flow of treatment gas; and
the cushion cover of claim 1.

10. The patient interface device of claim 9, wherein the patient interface device is a nasal/oral type patient interface device.

11. The patient interface device of claim 9, wherein the patient interface device is a nasal type patient interface device.

12. The patient interface device of claim 1, wherein the first portion and the second portion are each composed of silicone, wherein at least a portion of the first portion is coated with a fabric material coated, and wherein the second portion is not coated with the fabric material.

13. The patient interface device of claim 1, wherein the first portion and the second portion are each composed of silicone, wherein the first portion is composed of silicone including an additional additive material and the second portion is composed of silicone not including the additional additive material, and wherein the additive material includes at least one of a hydrophilic additive material, a wrinkle-reducing additive material, and a scented additive material.
